(19) United States
(12) Patent Application Publication
(10) Pub. No.: US 2023/0417742 A1
(43) Pub. Date: Dec. 28, 2023

(54) IMPROVED ASSAYS TO DETECT NUCLEOSOME MODIFICATIONS USING ANTIBODY-TARGETED ENZYME DIGESTION

(71) Applicant: EPICYPHER, INC., Durham, NC (US)

(72) Inventors: Matthew R. Marunde, Carrboro, NC (US); Nathan W. Hall, Chapel Hill, NC (US); Zu-Wen Sun, Brentwood, TN (US); Martis W. Cowles, Chapel Hill, NC (US); Andrea L. Johnstone, Apex, NC (US); Michael-Christopher Keogh, Cambridge, MA (US); Ellen Weinzapfel, Durham, NC (US); Kelsey Noll, Durham, NC (US)

(21) Appl. No.: 18/251,279

(22) PCT Filed: Nov. 2, 2021
(86) PCT No.: PCT/US2021/057680

§ 371 Date: May 1, 2023

Related U.S. Application Data
(60) Provisional application No. 63/108,578, filed on Nov. 2, 2020.

(51) Int. Cl. G01N 33/53; G01N 33/68

(52) U.S. Cl. CPC G01N 33/5308; G01N 33/6875

(57) ABSTRACT

This present invention relates to methods for improved assays to quantify the level of chromatin targets from biological samples. These assays can be used for the detection of global levels of epigenetic modifications (e.g., histone and DNA modifications), chromatin associated proteins, or chromatin associated ribonucleic acids (RNA). The invention further relates to assay kits that include the reagents needed to prepare biological samples and perform said improved chromatin assays.

### STATEMENT OF PRIORITY

This application claims the benefit of U.S. Provisional Application Ser. No. 62/108,578, filed Nov. 2, 2020, the entire contents of which are incorporated by reference herein.

### FIELD OF INVENTION

This present invention relates to methods for improved assays to quantify the level of chromatin targets from biological samples. These assays can be used for the detection of global levels of epigenetic modifications (e.g., histone and DNA modifications), chromatin associated proteins, or chromatin associated ribonucleic acids (RNA). The invention further relates to assay kits that include the reagents needed to prepare biological samples and perform said improved chromatin assays.

### BACKGROUND OF INVENTION

Nucleosomes are the fundamental repeating units of chromatin, made of 147 base pairs of DNA wrapped around a histone octamer (containing 2 copies each of the core histones (H2A, H2B, H3 and H4)) (Margueron et al., Nat. Rev. Genet. 11(4):285 (2010)). Changes in chromatin structure and function regulate diverse cellular activities including gene expression and cell differentiation (Brown et al., Hum. Mol. Genet. 21(R1):R90 (2012); Lahtz et al., J Mol. Cell. Biol. 3(1):51 (2011); Lunyak et al., Hum. Mol. Genet. 17(R1):R28 (2008); Reik, Nature 447(7143):425 (2007)). These processes are mediated in part by reversible epigenetic modifications, including both histone post-translational modifications (PTMs; e.g., lysine methylation and acetylation) and DNA methylation (e.g., 5-methylcytosine and 5-hydroxymethylcytosine), which have direct effects or are “read” by effector binding proteins to transduce specific downstream signaling pathways. As such, aberrant epigenetic regulation of chromatin has been linked to multiple human diseases, ranging from neurodegeneration (Landgrave-Gomez et al., Front. Cell. Neurosci. 9:58 (2015)) and metabolic syndrome (DelCurto et al., Curr. Opin. Clin. Nutr. Metab. Care 16(4):385 (2013); Wang et al., Antioxid. Redox Signal 17(2):282 (2012)) to cancer (Chopra et al., Cancer Genet. 208(5):192 (2015); Greenblatt et al., Leukemia 28(7):1396 (2014); Gajer et al., Oncogenesis 4:e137 (2015); Witt et al., Curr. Pharm. Des. 15(4):436 (2009); Hanmod et al., Pediatr. Blood Cancer 62(1):52 (2015); Kobayashi et al., Oncogene 32(21):2640 (2013)). Significantly, histone PTMs and chromatin associated proteins are highly druggable making them exceptional therapeutic targets to treat myriad human diseases/disorders (Arrowsmith et al., Nat. Rev. Drug Discov. 11(5):384 (2012)). Moreover, histone and DNA modifications are an emerging class of cancer biomarkers that may be useful for early disease detection and prognosis as well as informing personalized treatment strategies (Khan et al., World J. Biol. Chem. 6(4):333 (2015); Chervona et al., Am. J. Cancer Res. 2(5):589 (2012)).

Several methods have been developed to measure epigenetic modifications and chromatin associated proteins from biological samples (Sidoli et al., J. Vis. Exp. 2016(111); Onder et al., Expert Rev. Proteomics 12(5):499 (2015); Machleidt et al., J Biomol. Screen. 16(10):1236 (2011)). For example, Chromatin ImmunoPrecipitation (ChIP) uses modification specific antibodies for nucleosome enrichment followed by next-generation sequencing, generating maps of histone PTM or chromatin-bound protein localization across the genome; however, these assays exhibit low sensitivity and can be highly unreliable. Next-generation genomic mapping assays use “chromatin tethering” methods, which affix enzymes to specific genomic regions, resulting in labeling/release and selective analysis of target material (e.g. DamID, ChIC, CUT&RUN, and CUT&Tag) (van Steensel and Henikoff 2000, Schmid, Durussel et al. 2004, Meers, Bryson et al. 2019, Henikoff and Henikoff 2020). CUT&RUN (Cleavage Under Targets and Release Using Nuclease) uses a target-specific antibody (e.g., histone PTM or chromatin bound protein) to tether pAG-MNase (a fusion of protein A-protein G and micrococcal nuclease) to target-enriched sites in intact cells, which is then activated by the addition of calcium to cleave local DNA, which diffuses from the uncut pool. The CUT&RUN protocol is further streamlined by using a solid support to adhere cells/nuclei to lectin-coated magnetic beads and facilitate separation of the cut and uncut pools. In this manner background is dramatically reduced and the protocol can generate reliable genomic mapping data using as few as 100 cells (Skene, Henikoff et al. 2018, Meers, Bryson et al. 2019) and 3 million reads. While useful for genomic mapping, chromatin tethering methods are not yet compatible with detection of global nucleosome levels. Other methods for quantifying nucleosomes from biological samples use two detection reagents (or antibodies), wherein a first detection reagent captures a nucleosome (similar to above) and a second detection reagent is used for target epitope detection (e.g., sandwich ELISA (Enzyme Linked ImmunoSorbent Assay); WO 2003/070894, WO 2005/019826). ELISA is commonly used to quantify global levels of histone or DNA modifications from biological samples, with specific assays developed to directly quantify specific modifications on DNA, histones or nucleosomes using various antibody capture approaches. Concerning histone PTMs, nucleosome-based detection is superior to histone-based for three major reasons:
1) Nucleosome-based methods do not require acid-extraction steps, which are laborious and introduce variability.
2) Nucleosome-based assays allow quantification of combinatorial modifications (e.g., histone-histone, DNA-DNA, or histone-DNA combinations), impossible to monitor using histone subunits or DNA alone.
3) Nucleosome-based assays allow for use of nucleosome controls, which provide reliable quantification from biological samples, including liquid biopsy (see below).

Recently, semi-synthetic recombinant nucleosomes carrying histone or DNA modifications have been developed as assay standards, providing reliable quantitative controls for immunoassays that measure global modification levels (such as ELISA; (Thalin, Aguilera et al. 2020) and WO 2019/169263) or their genomic distribution (such as ChIP-seq; (Grzybowski, Chen et al. 2015, Shah, Grzybowski et al. 2018, Grzybowski, Shah et al. 2019) and WO 2013/184930, WO 2015/117145, WO 2020/140082, WO 2020/132388). Nucleosome-based controls are especially useful for liquid biopsy assays as they can be faithfully recovered from patient plasma vs. histone-based controls, which are not reliably recovered from plasma samples ((Thalin, Aguilera et al. 2020) and WO 2019/169263). In addition, modified recombinant nucleosomes also provide useful tools for antibody validation. Indeed, >70% of commercial chromatin targeting antibodies exhibit unacceptable cross-reactivity and/or efficiency when targeting nucleosomes (Shah, Grzybowski et al. 2018). This massive problem with antibody specificity is likely caused by the dramatic difference in antibody performance when targeting histone peptides (the current gold-standard validation approach) vs. nucleosome substrates, suggesting that nucleosome-based technologies are the future of PTM antibody profiling. Together these advances have enabled the development of specific and quantitative assays to measure histone and/or DNA modifications from biological samples.

Despite progress, the development of reliable assays that measure nucleosomes, nucleosome modifications (DNA/histone PTMs), or chromatin associated proteins from cellular chromatin or from biological fluids (e.g., circulating cell-free nucleosomes or cell-free DNA) can be challenging, caused by limitations in sample processing and/or nucleosome capture/detection. Indeed, current state-of-the-art ELISA use a sandwich ELISA format, wherein a first antibody captures a nucleosome by a first epitope; this could be an unmodified histone, modified histone, unmodified DNA, modified DNA, chromatin associated protein, or RNA. A second antibody is then used to detect the nucleosome by binding to a different second epitope; this could also be an unmodified histone, modified histone, unmodified DNA, modified DNA, chromatin associated protein, or RNA. There are several challenges with current assays. First, processing cellular chromatin into mono- and/or polynucleosome fragments is time-consuming and a major source of variability. This is particularly important for cellular assays, in which chromatin fragments need to be liberated from chromatin prior to analysis. Indeed, current leading methods used to fragment chromatin into mono- and/or polynucleosome fragments (or subnucleosomal fragments in the case of chromatin associated proteins) use mechanical (via sonication) or enzymatic (via MNase) digestion, both of which require extensive optimization for each cell/sample type. While technically straightforward, sonication typically requires fixation, which result in biased cleavage of DNA and high variation between samples (Baranello, Kouzine et al. 2016). Enzymatic digestion by DNA cleaving enzymes (e.g., MNase) also requires extensive optimization and can result in biased cleavage. Further, over digestion can result in nucleosome instability (Xi, Yao et al. 2011, Zhang and Pugh 2011). Second, identifying antibody combinations that generate high signal-to-noise in sandwich ELISAs can be notoriously difficult. Moreover, chromatin provides an extra layer of complexity as chromatin is heavily modified, and antibody binding can be precluded by the presence of nearby histone and/or DNA modifications. Thus, antibodies that bind unmodified histone or DNA modifications (generally used for nucleosome detection) are very challenging to validate as it is difficult with current tools to test the impact of every possible combinatorial modification state on antibody binding. One potential solution to this issue is to use a single antibody assay format. These types of assays are known in the art but can lack sensitivity and reliability. For example, it is possible to capture a chromatin fragment using an antibody to a chromatin target epitope (as described above), followed by analyzing associated DNA. This could be done by various assay readouts, including the use of intercalating dyes. However, these assays fail to account for differences in length of nucleosomal DNA (cell derived or circulating nucleosomes) and thus are challenging to quantify, normalize, and compare across samples. Treating samples with MNase to standardize DNA length is not ideal, as enzymatic digestion can result in nucleosome instability, result in DNA degradation and loss of signal (Chereji, Bryson et al. 2019). Given the above, there is a need in the art for improved chromatin-based immunoassays that detect and measure histone and/or DNA modifications from biological samples. The development of such assays would be widely used for chromatin research and diagnostic assay applications.

### SUMMARY OF INVENTION

This present invention relates to methods for improved assays that detect global levels of nucleosome modifications and/or associated proteins from biological samples, including cellular chromatin and cell-free DNA. These assays use a detection reagent to bind chromatin from a biological sample. Next, chromatin is treated with an enzyme that is targeted to the detection reagent and digests nearby DNA. The cleaved chromatin fragment (e.g., a nucleosome composed of ˜150 base pairs of DNA protected from enzyme digestion by tight association with the histone octamer and/or a protein-DNA fragment composed of <150 bp of DNA protected from enzyme digestion by tight association with the chromatin associated protein), is then quantified by measuring the amount of DNA. Targeted enzymatic digestion is key to this approach, as it provides fine-tuned digestion of nucleosomal DNA (i.e., mitigates potential over digestion by untargeted enzymatic digestion), which is essential to standardize DNA length per nucleosome (or captured protein-DNA fragment) for reliable assay quantification. This approach also allows for the development of assays using a single detection reagent (vs. current sandwich ELISA approaches), streamlining assay development and mitigating compatibility issues or impacts of existing modifications that complicate sandwich ELISA formats. Further, this approach provides an efficient assay workflow for analyzing chromatin directly from cells, as chromatin carrying specific proteins and/or modifications can be selectively digested from chromatin without the need of sonication or global enzymatic digestion.

Thus, one aspect of the invention relates to a method for measuring the total amount of a chromatin target epitope in a biological sample, the method comprising:
a. obtaining a biological sample comprising chromatin;
b. contacting the chromatin with a detection reagent that binds a chromatin target epitope to form a complex;
c. contacting the complex with a detection reagent-binding agent linked to an inactive enzyme;
d. activating the enzyme to digest the chromatin in a targeted manner to release chromatin fragments;
e. trimming DNA associated with the released chromatin to produce a uniform length;
f. isolating the released chromatin fragments; and
g. quantifying the amount of DNA associated with the released chromatin fragment;
thereby measuring the total amount of the chromatin target epitope in the biological sample;
wherein the detection reagent is the only chromatin binding agent used in the method.

Another aspect of the invention relates to a method for measuring the total amount of a chromatin target epitope in a biological sample, the method comprising:
a. obtaining a biological sample comprising chromatin;
b. obtaining reference nucleosomes comprising the chromatin target epitope to form a reference sample;
c. contacting each of the chromatin and the reference sample with a detection reagent that binds a chromatin target epitope to form a complex;
d. contacting the complex with a detection reagent-binding agent linked to an inactive enzyme;
e. activating the enzyme to digest the chromatin and the reference sample in a targeted manner to release chromatin fragments;
f. trimming DNA associated with the released chromatin fragments to produce a uniform length;
g. isolating the released chromatin fragments from the chromatin and the reference sample; and
h. quantifying the amount of DNA associated with the released chromatin fragments from the chromatin and the reference sample;
thereby quantifying the level of the chromatin target epitope in the biological sample;
wherein the detection reagent is the only chromatin binding agent used in the method.

A further aspect of the invention relates to a method for measuring the total amount of a chromatin target epitope in a biological sample, the method comprising:
a. obtaining a biological sample comprising chromatin;
b. binding the biological sample to a solid support;
c. contacting the chromatin with a detection reagent that binds a chromatin target epitope to form a complex;
d. contacting the complex with a detection reagent-binding agent linked to an inactive enzyme;
e. activating the enzyme to digest the chromatin in a targeted manner to release chromatin fragments from the solid support;
f. trimming DNA associated with the released chromatin fragments to produce a uniform length;
g. isolating the released chromatin fragments; and
h. quantifying the amount of DNA associated with the released chromatin fragments;
thereby measuring the total amount of the chromatin target epitope in the biological sample;
wherein the detection reagent is the only chromatin binding agent used in the method.

An additional aspect of the invention relates to a method for measuring the total amount of a chromatin target epitope in a biological sample, the method comprising:
a. obtaining a biological sample comprising chromatin;
b. obtaining reference nucleosomes comprising the chromatin target epitope to form a reference sample;
c. binding the biological sample and the reference sample to a solid support;
d. contacting each of the chromatin and the reference sample with a detection reagent that binds a chromatin target epitope to form a complex;
e. contacting the complex with a detection reagent-binding agent linked to an inactive enzyme;
f. activating the enzyme to digest the chromatin and the reference sample in a targeted manner to release chromatin fragments from the solid support;
g. trimming DNA associated with the released chromatin fragments to produce a uniform length;
h. isolating the released chromatin fragments from the chromatin and the reference sample; and
i. quantifying the amount of DNA associated with the released chromatin fragments from the chromatin and the reference sample;
thereby quantifying the level of the chromatin target epitope in the biological sample;
wherein the detection reagent is the only chromatin binding agent used in the method.

Another aspect of the invention relates to a method for measuring the total amount of a chromatin target epitope in a biological sample, the method comprising:
a. obtaining a biological sample comprising cell-free DNA;
b. contacting the cell-free DNA with a detection reagent that binds a chromatin target epitope to form a complex;
c. contacting the complex with a detection reagent-binding agent linked to an inactive enzyme;
d. activating the enzyme to digest the cell-free DNA in a targeted manner to produce digested chromatin fragments;
e. trimming DNA associated with the digested chromatin fragments to produce a uniform length; and
f. quantifying the amount of DNA associated with the digested chromatin fragments;
thereby measuring the total amount of the chromatin target epitope in the biological sample;
wherein the detection reagent is the only chromatin binding agent used in the method.

A further aspect of the invention relates to a method for measuring the total amount of a chromatin target epitope in a biological sample, the method comprising:
a. obtaining a biological sample comprising cell-free DNA;
b. obtaining reference nucleosomes comprising the chromatin target epitope to form a reference sample;
c. contacting each of the cell-free DNA and the reference sample with a detection reagent that binds a chromatin target epitope to form a complex;
d. contacting the complex with a detection reagent-binding agent linked to an inactive enzyme;
e. activating the enzyme to digest the cell-free DNA and the reference sample in a targeted manner to produce digested chromatin fragments;
f. trimming DNA associated with the digested chromatin fragments to produce a uniform length; and
g. quantifying the amount of DNA associated with the digested chromatin fragments from the cell-free DNA and the nucleosome reference sample;
thereby quantifying the level of the chromatin target epitope in the biological sample;
wherein the detection reagent is the only chromatin binding agent used in the method.

An additional aspect of the invention relates to a method for measuring the total amount of a chromatin target epitope in a biological sample, the method comprising:
a. obtaining a biological sample comprising cell-free DNA;
b. binding the biological sample to a solid support;
c. contacting the cell-free DNA with a detection reagent that binds a chromatin target epitope to form a complex;
d. contacting the complex with a detection reagent-binding agent linked to an inactive enzyme;
e. activating the enzyme to digest the cell-free DNA in a targeted manner to produce digested chromatin fragments;
f. trimming DNA associated with the digested chromatin fragments to produce a uniform length; and
g. quantifying the amount of DNA associated with the digested chromatin fragments;
thereby measuring the total amount of the chromatin target epitope in the biological sample;
wherein the detection reagent is the only chromatin binding agent used in the method.

Another aspect of the invention relates to a method for measuring the total amount of a chromatin target epitope in a biological sample, the method comprising:
a. obtaining a biological sample comprising cell-free DNA;
b. obtaining reference nucleosomes comprising the chromatin target epitope to form a reference sample;
c. binding the biological sample and the reference sample to a solid support;
d. contacting each of the cell-free DNA and the reference sample with a detection reagent that binds a chromatin target epitope to form a complex;
e. contacting the complex with a detection reagent-binding agent linked to an inactive enzyme;
f. activating the enzyme to digest the cell-free DNA and the reference sample in a targeted manner to produce chromatin fragments;
g. trimming DNA associated with the digested chromatin fragments to produce a uniform length; and
h. quantifying the amount of DNA associated with the digested chromatin fragments from the chromatin and the reference sample;
thereby quantifying the level of the chromatin target epitope in the biological sample;
wherein the detection reagent is the only nucleosome binding agent used in the method.

A further aspect of the invention relates to kits for carrying out the methods of the invention.

These and other aspects of the invention are set forth in more detail in the description of the invention below.

### BRIEF DESCRIPTION OF THE DRAWINGS

FIG. 1 shows a schematic overview of workflow using pAG-MNase to standardize DNA size followed by DNA quantification. Nucleosomes are first captured using an antibody to a nucleosome modification or chromatin binding protein. pAG-Mnase is then added to the samples and activated to cleave nearby DNA, resulting in standardization of captured nucleosomal DNA length. Samples are treated with Proteinase K to remove histone proteins. Finally, DNA is quantified using an intercalating dye.

FIGS. 2A-2B show targeted cleavage via pAG-MNase provides an improved digestion method to standardize DNA vs traditional MNase treatment. (A-B) Protein A beads bound to an anti-H3K4me2 (ThermoFisher 701764) were used to capture H3K4me2 nucleosomes assembled with either 147 bp or 250 bp DNA. 147 bp or 250 bp free DNA was used as a control. (A) MNase (Worthington LS004798) and (B) pAG-MNase (EpiCypher 15-1016) were titrated at various concentrations, activated with 2 mM CaCl₂), and incubated for 30 minutes at room temperature. Nucleosomal DNA was then liberated via Proteinase K digest and quantified using PicoGreen® fluorescence.

FIG. 3 shows removal of histone proteins via Proteinase K digestion is required for accurate detection using the dsDNA intercalating molecule PicoGreen®. Either 147 bp DNA or nucleosomes containing 147 bp were exposed to the presence (+) or absence (−) of Proteinase K and incubated for 15 minutes at 37° C. DNA was then treated with PicoGreen® and quantified by measuring fluorescence.

FIG. 4 shows an overview of magnetic bead and plate adsorption immuno-capture methods. The capture antibody of choice can be immobilized in a variety of methods in order to reliably capture exogenous (spike-in standard) and endogenous forms of respective antigen. Utilizing magnetic Dynabeads impregnated with either Protein A (for capture antibody heavy/light chain interaction) or Streptavidin (for interaction with biotinylated capture antibody) constitutes an indirect antigen capture method. Conversely, employing a carboxylated bead set allows the chemical conjugation of a chosen capture antibody and adsorption of the capture antibody to a well surface constituting a direct antigen capture method.

FIGS. 5A-5B show utilization of a plate adsorption method (a direct antigen capture method) in a liquid biopsy setting. (A-B) Abundance of cell-free nucleosomes bearing H3K4me3 (A) and H3K27me3 (B) was determined in rheumatoid arthritis (RA) and healthy plasma. Capture antibodies (anti-H3K4me3 or anti-H3K27me3) were first adsorbed to a 96-well microtiter plate. Next, wells were treated with plasma samples. Nucleosomes bound to the plate were digested with pAG-MNase, treated with Proteinase K, treated with PicoGreen®, and DNA was quantified via PicoGreen® fluorescence. Similar assays were performed in plasma spiked with various amounts of H3K4me3 or H3K27em3 nucleosomes to generate a standard curve (not shown). Recovered Fluorescent Intensity values were compared to the standard curve to determine the recovery of endogenous H3K4me3 or H3K27me3 in RA and healthy plasma samples. An increase in H3K27me3 nucleosomes was observed in two of the RA samples compared to the controls (B). No major difference in H3K4me3 nucleosomes was observed between RA and healthy controls (A).

FIGS. 6A-6B show targeted cleavage of nucleosomes by pAG-MNase can be used to quantify global levels of histone PTMs directly from cells. (A) K562 cells were permeabilized with 0.01% digitonin and incubated overnight with antibodies to Rb IgG, H3K4me3, or H3K27me3. pAG-MNase was then added to each reaction and activated with 2 mM CaCl₂. Released DNA fragments were purified using carboxylated beads, treated with Proteinase K and PicoGreen®, and then quantified using PicoGreen® fluorescence. (B) In separate reactions, nucleosomes carrying unmodified, H3K4me3, or H3K27me3 nucleosomes were bound to a solid support and processed as described above to generate a standard curve. Assays in (A) and (B) were performed on a Tecan Freedom EVO robotics platform. (B) shows a comparison of the DNA quantification from the standard curve prepared manually versus automated.

FIG. 7 shows workflow for cell-based assays: a. Detection reagent binds to target; b. pAG-MNase binds antibody; c. Ca²⁺ activates MNase to cleave and release target nucleosomes; d. In combinatorial assays, second mark selected by plate capture; e. DNA is released from nucleosomes by proteinase-K treatment and detected via PicoGreen® (star).

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of nucleosomes, and transiently and stably transfected cells. Such techniques are known to those skilled in the art. See, e.g., SAMBROOK et al., MOLECULAR CLONING: A LABORATORY MANUAL 4th Ed. (Cold Spring Harbor, N Y, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

The term “consisting essentially of” as used herein in connection with a nucleic acid, protein means that the nucleic acid or protein does not contain any element other than the recited element(s) that significantly alters (e.g., more than about 1%, 5% or 10%) the function of interest of the nucleic acid or protein.

As used herein, the term “polypeptide” encompasses both peptides and proteins, unless indicated otherwise.

A “nucleic acid” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

By “substantially retain” a property, it is meant that at least about 75%, 85%, 90%, 95%, 97%, 98%, 99% or 100% of the property (e.g., activity or other measurable characteristic) is retained.

The term “epitope” refers to any site on a biomolecule that can evoke binding of an detection reagent. The detection reagent might recognize a linear sequence of a biomolecule or biomolecule fragment, the shape of biomolecule or biomolecule fragment, a chemo-physical property of a biomolecule or biomolecule fragment, or a combination of these.

“Amino acids” may be referred to herein by either their commonly known three letter symbols or by the one-letter symbols recommended by the IUPAC-IUB Biochemical Nomenclature Commission. Amino acid residues in proteins or peptides are abbreviated as follows: phenylalanine is Phe or F; leucine is Leu or L; isoleucine is Ile or I; methionine is Met or M; valine is Val or V; serine is Ser or S; proline is Pro or P; threonine is Thr or T; alanine is Ala or A; tyrosine is Tyr or Y; histidine is His or H; glutamine is Gln or Q; asparagine is Asn or N; lysine is Lys or K; aspartic acid is Asp or D; glutamic Acid is Glu or E; cysteine is Cys or C; tryptophan is Trp or W; arginine is Arg or R; and glycine is Gly or G.

The term “amino acid” refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., a carbon that is bound to a hydrogen, a carboxyl group, an amino group, and an R group, such as, homoserine, norleucine, methionine sulfoxide, and methionine methyl sulfonium. Such analogs have modified R groups (such as, norleucine) or modified peptide backbones, but retain the same basic chemical structure as a naturally occurring amino acid.

As to amino acid sequences, one of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are known to those of skill in the art. Such conservatively modified variants are in addition to and do not exclude polymorphic variants, interspecies homologs/orthologs, and alleles of the agents described herein.

An “antigen” as used herein may be any structure which is recognized by an antibody or for which recognizing antibodies (or analogous affinity reagents such as aptamers or panned phage) can be raised. In certain embodiments, antigens may comprise a single amino acid residue or an amino acid fragment of 2 or more residues. In certain embodiments, antigens may comprise modifications of an amino acid, such as acetylation, methylation (e.g., mono-, di-, tri-, symmetric-, asymmetric-), phosphorylation, ubiquitination (e.g., mono-, di-, tri-, poly-), sumoylation, ADP-ribosylation, citrullination, biotinylation, and cis-trans isomerization. In certain embodiments, antigens may comprise nucleotide modifications, such as 5-methylcytosine. In other embodiments, antigens may comprise specific mutations, such as point mutations. In yet other embodiments, antigens may comprise wild-type amino acid sequences or nucleotide sequences.

The term “post-translational modification” refers to any modification of a natural or non-natural amino acid that occurs or would occur to such an amino acid after it has been incorporated into a polypeptide chain in vivo or in vitro. Such modifications include, but are not limited to, acylation (e.g., acetyl-, butyryl-, crotonyl-), methylation (e.g., mono-, di-, tri-), phosphorylation, ubiquitination (e.g., mono-, di-, tri-, poly-), sumoylation, ADP-ribosylation, citrullination, biotinylation, and cis-trans isomerization. Such modifications may be introduced synthetically, e.g., chemically, during polypeptide synthesis or enzymatically after polypeptide synthesis or polypeptide purification.

The term “post-transcriptional modification” refers to any modification of a natural or non-natural nucleotide that occurs or would occur to such a nucleotide after it has been incorporated into a polynucleotide chain in vivo or in vitro. Such modifications include, but are not limited to, 5-methylcyosine, 5-hydroxymethylcytosine, 5,6-dihydrouracil, 7-methylguanosine, xanthosine, and inosine.

The term “cell-free DNA” refers to chromatin found outside of a cell. The cell-free DNA may be associated with various proteins such as histones (e.g., nucleosomes) or other chromatin associated proteins, which shield DNA from digestion by various enzymes, such as micrococcal nuclease (MNase). Cell free DNA can be found in any type of biological fluid (e.g., blood, serum, plasma, urine, saliva, semen, prostatic fluid, nipple aspirate, lachrymal fluid, perspiration, feces, cheek swabs, cerebrospinal fluid, cell lysate samples, amniotic fluid, gastrointestinal fluid, biopsy tissue, or lymphatic fluid, etc.) or media used to culture cells, tissues, or organs.

The present invention relates to methods for improved assays that detect global levels of nucleosome modifications and/or chromatin associated proteins from biological samples, including cellular chromatin and cell-free DNA. These assays use a detection reagent to bind chromatin from a biological sample. Next, samples are treated with an enzyme that is targeted to the detection reagent and digests nearby DNA. The cleaved chromatin fragment is then quantified by measuring the amount of DNA. For nucleosomes, the cleaved fragments are composed of ˜150 base pairs of DNA protected from enzyme digestion by tight association with the histone octamer and/or associated proteins, whereas for chromatin associated proteins, the cleaved fragments are generally <150 bp and could be, e.g., between 10-50 bp, 50-100 bp, or 100-150 bp. Targeted enzymatic digestion is key to this approach, as it provides fine-tuned digestion of chromatin DNA (i.e., mitigates potential over digestion by untargeted enzymatic digestion), which is essential to standardize DNA length per chromatin fragment for reliable assay quantification. This approach also allows for the development of assays using a single detection reagent (vs. current sandwich ELISA approaches), streamlining assay development and mitigating compatibility issues or impacts of existing modifications that complicate sandwich ELISA formats. Further, this approach provides an efficient assay workflow for analyzing chromatin directly from cells, as nucleosomes carrying specific modifications can be selectively digested from chromatin without the need of sonication or global enzymatic digestion.

The methods of the invention are described in the following general protocols.

The invention contains the following method to analyze chromatin from biological samples:
a sample is prepared containing chromatin;
a detection reagent is added to the sample that binds a chromatin target epitope;
an inactive targeted enzyme is added to the sample that binds to the detection reagent;
targeted enzyme is then activated to digest nearby DNA in the sample;
digested chromatin fragments are then purified from the sample and quantified using an assay that measures nucleic acid.

The invention contains the following method to analyze chromatin from biological samples:
a sample is prepared containing chromatin;
recombinant nucleosomes carrying a target epitope (e.g., histone or DNA modification) are prepared at a single concentration or a range of concentrations to make an assay standard;
a detection reagent is added to sample and assay standard that binds a chromatin target epitope;
an inactive targeted enzyme is added to the sample and assay standard that binds to the detection reagent;
targeted enzyme is then activated to digest nearby chromatin DNA in sample and assay standard;
digested chromatin fragments are then purified from the sample and assay standard and quantified using an assay that measures nucleic acid;
recovered levels from the assay standard are used to quantify chromatin target epitope levels in the biological sample.

The invention contains the following method to analyze chromatin from biological samples:
a sample containing chromatin is bound to a solid support;
a detection reagent is added to sample that binds a chromatin target epitope;
an inactive targeted enzyme is added to the sample that binds to the detection reagent;
targeted enzyme is then activated to cleave targeted chromatin from the solid support;
targeted chromatin fragments are then purified from the sample and quantified using an assay that measures nucleic acid.

The invention contains the following method to analyze chromatin from biological samples:
a sample containing chromatin is bound to a solid support;
recombinant nucleosomes carrying a target epitope (e.g., histone or DNA modification) are prepared at a single concentration or a range of concentrations to make an assay standard and bound to a solid support;
a detection reagent is added to sample and assay standard that binds a chromatin target epitope;
an inactive targeted enzyme is added to the sample and assay standard that binds to the detection reagent;
targeted enzyme is then activated to cleave targeted chromatin from the solid support;
targeted chromatin fragments are then purified from the sample and assay standard and quantified using an assay that measures nucleic acid;
recovered levels from the assay standard are used to quantify chromatin target epitope levels in the biological sample.

The invention contains the following method to analyze chromatin from biological samples:
a sample is prepared containing chromatin;
a detection reagent coupled to a magnetic bead (i.e., solid support) is added to the sample that binds a chromatin target epitope;
an inactive targeted enzyme is added to the sample that binds to the detection reagent;
targeted enzyme is then activated to digest nearby chromatin DNA in the sample;
digested chromatin fragments are then purified from the sample and quantified using an assay that measures nucleic acid.

The invention contains the following method to analyze chromatin from biological samples:
chromatin is prepared from a biological sample;
recombinant nucleosomes carrying a target epitope (e.g., histone or DNA modification) are prepared at a single concentration or a range of concentrations to make an assay standard and bound to a solid support;
a detection reagent coupled to a magnetic bead (i.e., solid support) is added to the sample and assay standard that binds a chromatin target epitope;
an inactive targeted enzyme is added to the sample and assay standard that binds to the detection reagent;
targeted enzyme is then activated to digest nearby chromatin DNA in sample and assay standard;
digested chromatin fragments are then purified from the sample and assay standard and quantified using an assay that measures nucleic acid;
recovered levels from the assay standard are used to quantify target epitope levels in the biological sample.

Thus, one aspect of the invention relates to a method for measuring the total amount of a chromatin target epitope in a biological sample, the method comprising:
a. obtaining a biological sample comprising chromatin;
b. contacting the chromatin with a detection reagent that binds a chromatin target epitope to form a complex;
c. contacting the complex with a detection reagent-binding agent linked to an inactive enzyme;
d. activating the enzyme to digest the chromatin in a targeted manner to release chromatin fragments;
e. trimming DNA associated with the released chromatin fragments to produce a uniform length;
f. isolating the released chromatin fragments; and
g. quantifying the amount of DNA associated with the released chromatin fragments;
thereby measuring the total amount of the chromatin target epitope in the biological sample;
wherein the detection reagent is the only chromatin binding agent used in the method.

Another aspect of the invention relates to a method for measuring the total amount of a chromatin target epitope in a biological sample, the method comprising:
a. obtaining a biological sample comprising chromatin;
b. obtaining reference nucleosomes comprising the chromatin target epitope to form a reference sample;
c. contacting each of the chromatin and the reference sample with a detection reagent that binds a chromatin target epitope to form a complex;
d. contacting the complex with a detection reagent-binding agent linked to an inactive enzyme;
e. activating the enzyme to digest the chromatin and the reference sample in a targeted manner to release chromatin fragments;
f. trimming DNA associated with the released chromatin fragments to produce a uniform length;
g. isolating the released chromatin fragments from the chromatin and the reference sample; and
h. quantifying the amount of DNA associated with the released chromatin fragments from the chromatin and the reference sample;
thereby quantifying the level of the chromatin target epitope in the biological sample;
wherein the detection reagent is the only chromatin binding agent used in the method.

A further aspect of the invention relates to a method for measuring the total amount of a chromatin target epitope in a biological sample, the method comprising:
a. obtaining a biological sample comprising chromatin;
b. binding the biological sample to a solid support;
c. contacting the chromatin with a detection reagent that binds a chromatin target epitope to form a complex;
d. contacting the complex with a detection reagent-binding agent linked to an inactive enzyme;
e. activating the enzyme to digest the chromatin in a targeted manner to release chromatin fragments from the solid support;
f. trimming DNA associated with the released chromatin fragments to produce a uniform length;
g. isolating the released nucleosomes; and
h. quantifying the amount of DNA associated with the released chromatin fragments;
thereby measuring the total amount of the chromatin target epitope in the biological sample;
wherein the detection reagent is the only chromatin binding agent used in the method.

An additional aspect of the invention relates to a method for measuring the total amount of a chromatin target epitope in a biological sample, the method comprising:
a. obtaining a biological sample comprising chromatin;
b. obtaining reference nucleosomes comprising the chromatin target epitope to form a reference sample;
c. binding the biological sample and the reference sample to a solid support;
d. contacting each of the chromatin and the reference sample with a detection reagent that binds a chromatin target epitope to form a complex;
e. contacting the complex with a detection reagent-binding agent linked to an inactive enzyme;
f. activating the enzyme to digest the chromatin and the reference sample in a targeted manner to release chromatin fragments from the solid support;
g. trimming DNA associated with the released chromatin fragments to produce a uniform length;
h. isolating the released chromatin fragments from the chromatin and the reference sample; and
i. quantifying the amount of DNA associated with the released chromatin fragments from the chromatin and the reference sample;
thereby quantifying the level of the chromatin target epitope in the biological sample;
wherein the detection reagent is the only chromatin binding agent used in the method.

Another aspect of the invention relates to a method for measuring the total amount of a chromatin target epitope in a biological sample, the method comprising:
a. obtaining a biological sample comprising cell-free DNA;
b. contacting the cell-free DNA with a detection reagent that binds a chromatin target epitope to form a complex;
c. contacting the complex with a detection reagent-binding agent linked to an inactive enzyme;
d. activating the enzyme to digest the cell-free DNA in a targeted manner to produce digested chromatin fragments;
e. trimming DNA associated with the digested chromatin fragments to produce a uniform length; and
f. quantifying the amount of DNA associated with the digested chromatin fragments;
thereby measuring the total amount of the chromatin target epitope in the biological sample;
wherein the detection reagent is the only chromatin binding agent used in the method.

A further aspect of the invention relates to a method for measuring the total amount of a chromatin target epitope in a biological sample, the method comprising:
a. obtaining a biological sample comprising cell-free DNA;
b. obtaining reference nucleosomes comprising the chromatin target epitope to form a reference sample;
c. contacting each of the cell-free DNA and the reference sample with a detection reagent that binds a chromatin target epitope to form a complex;
d. contacting the complex with a detection reagent-binding agent linked to an inactive enzyme;
e. activating the enzyme to digest the cell-free DNA and the reference sample in a targeted manner to produce digested chromatin fragments;
f. trimming DNA associated with the digested chromatin fragments to produce a uniform length; and
g. quantifying the amount of DNA associated with the digested chromatin fragments from the cell-free DNA and the reference sample;
thereby quantifying the level of the chromatin target epitope in the biological sample;
wherein the detection reagent is the only chromatin binding agent used in the method.

An additional aspect of the invention relates to a method for measuring the total amount of a chromatin target epitope in a biological sample, the method comprising:
a. obtaining a biological sample comprising cell-free DNA;
b. binding the biological sample to a solid support;
c. contacting the cell-free DNA with a detection reagent that binds a chromatin target epitope to form a complex;
d. contacting the complex with a detection reagent-binding agent linked to an inactive enzyme;
e. activating the enzyme to digest the cell-free DNA in a targeted manner to produce digested chromatin fragments;
f. trimming DNA associated with the digested chromatin fragments to produce a uniform length; and
g. quantifying the amount of DNA associated with the digested chromatin fragments;
thereby measuring the total amount of the chromatin target epitope in the biological sample;
wherein the detection reagent is the only chromatin binding agent used in the method.

Another aspect of the invention relates to a method for measuring the total amount of a chromatin target epitope in a biological sample, the method comprising:
a. obtaining a biological sample comprising cell-free DNA;
b. obtaining reference nucleosomes comprising the chromatin target epitope to form a reference sample;
c. binding the biological sample and the reference sample to a solid support;
d. contacting each of the cell-free DNA and the reference sample with a detection reagent that binds a chromatin target epitope to form a complex;
e. contacting the complex with a detection reagent-binding agent linked to an inactive enzyme;
f. activating the enzyme to digest the cell-free DNA and the reference sample in a targeted manner to produce digested chromatin fragments;
g. trimming DNA associated with the digested chromatin fragments to produce a uniform length; and
h. quantifying the amount of DNA associated with the digested chromatin fragments from the cell-free DNA and the reference sample;
thereby quantifying the level of the chromatin target epitope in the biological sample.

In each of the methods of the invention, the individual steps may be carried out in a different order than recited and may be carried out simultaneously as long as the desired results are achieved. For example, the step of activating the enzyme to digest the chromatin or cell-free DNA and the step of trimming DNA associated with the digested chromatin fragments to produce a uniform length may be carried out simultaneously.

In each of the methods of the invention, the biological sample may be any sample from which nucleosomes or chromatin can be isolated. In some embodiments, the biological sample comprises a cell comprising chromatin. In some embodiments, the biological sample is chromatin isolated from the cell. In some embodiments, the biological sample is chromatin isolated from the cell and fragmented using mechanical or enzymatic means. In some embodiments, the biological sample is chromatin not isolated from the cell. In some embodiments, the biological sample is nuclei isolated from the cell. In some embodiments, the biological sample is chromatin isolated from the nucleus.

In some embodiments, a biological sample is composed of biological fluids, such as blood, serum, plasma, urine, saliva, semen, prostatic fluid, nipple aspirate, lachrymal fluid, perspiration, feces, cheek swabs, cerebrospinal fluid, cell lysate samples, amniotic fluid, gastrointestinal fluid, biopsy tissue, or lymphatic fluid. In some embodiments, a biological sample is composed of a portion of a tissue or organ, e.g., a biopsy or other clinical sample. In some embodiments, the biological sample is from a subject (e.g., a tissue or organ of the subject) having a disease or disorder associated with changes in one or more histone post-translational modifications, DNA modifications, chromatin associated proteins (e.g., transcription factors), or associated with mutations in histones, e.g., a diseased cell. The cells may be obtained from the diseased organ or tissue by any means known in the art, including but not limited to biopsy, aspiration, and surgery.

In other embodiments, the cells are not cells from a tissue or organ affected by a disease or disorder associated with changes in histone post-translational modifications, DNA modifications, chromatin associated proteins (e.g., transcription factors), or associated with mutations in histones. The cells may be, e.g., cells that serve as a proxy for the diseased cells. The cells may be cells that are more readily accessible than the diseased cells, e.g., that can be obtained without the need for complicated or painful procedures such as biopsies. Examples of suitable cells include, without limitation, peripheral blood mononuclear cells.

In some embodiments, the methods of the invention may be carried out on a small number of cells or nuclei, e.g., less than 100,000, 10,000, 100, 500, 100, or 10 cells or nuclei. In some embodiments, the methods are carried out on single cells or nuclei. A variety of highly scalable single cell assay workflows may be used with the methods, e.g., nano-well array (e.g., ICELL8 by Takara), micro-droplet (e.g., Chromium by 10× genomics), and combinatorial indexing.

In other embodiments, the biological sample comprises cell-free DNA (e.g., circulating nucleosomes), e.g., as released from dying cells. The cell-free DNA may be, e.g., from blood or from cells from a disease or disorder associated with epigenetic modifications. In certain embodiments, the biological sample is plasma, urine, saliva, stool, lymphatic fluid, or cerebrospinal fluid.

In some embodiments, the biological sample may be treated with an enzyme (e.g., an untargeted nuclease) to digest chromatin into smaller fragments (e.g., mono- and/or polynucleosome fragments) before treating with detection reagent and further digestion using a targeted nuclease.

The subject may be any subject for which the methods of the present invention are desired. In some embodiments, the subject is a mammal, e.g., a human. In some embodiments, the subject is a laboratory animal, e.g., a mouse, rat, dog, or monkey, e.g., an animal model of a disease. In certain embodiments, the subject may be one that has been diagnosed with or is suspected of having a disease or disorder. In some embodiments, the subject may be one that is at risk for developing a disease or disorder, e.g., due to genetics, family history, exposure to toxins, etc.

In some embodiments, the chromatin, cells, or nuclei from the biological sample is bound to a solid support. The chromatin, cells, or nuclei may be bound directly or indirectly to the solid support. The solid support may be coated with a reagent to help with binding the chromatin, cells, or nuclei, e.g., concanavalin A or streptavidin. Examples of solid supports include, without limitation, a bead, the well of a multiwell plate, a slide, etc. The bead may be composed of natural materials (e.g., alginate) or synthetic materials (e.g., polystyrene). In some embodiments, the bead is a magnetic bead that can be separated by exposure to a magnetic field. In some embodiments, the detection reagent is bound directly to the solid support and the chromatin, cells, or nuclei are bound indirectly to the solid support through the detection reagent.

The chromatin target epitope may be any histone modification, histone variant, histone mutation, unmodified histone, unmodified DNA, DNA modification of interest, and/or chromatin associated protein (e.g., a factor that directly or indirectly binds a histone, histone modification, DNA, or DNA modification). In some embodiments, the histone and/or DNA modification is selected from the group consisting of N-acetylation of serine and alanine; phosphorylation of serine, threonine and tyrosine; N-crotonylation, N-acylation of lysine; N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine, ADP-ribosylation of arginine, aspartic acid and glutamic acid; oncogenic K-to-M mutations, 5-methylcytosine, 5-hydroxymethylcytosine, 5-formylcytosine, 5-carboxylcytosine, 3-methylcytosine, 5,6-dihydrouracil, 7-methylguanosine, xanthosine, inosine, and any combination thereof.

Chromatin associated proteins include any protein that directly or indirectly interacts with chromatin, including transcription factors that bind directly to DNA and ‘reader’ proteins/enzymes that interact with and/or modify histones and/or DNA. The chromatin associated protein may be, without limitation, a transcription factor, a chromatin reader, a histone/DNA modifying enzyme, or a chromatin regulatory complex. Examples of transcription factors include, without limitation, those listed at: en.wikipedia.org/wiki/List_of_human_transcription_factors, incorporated by reference herein in its entirety. Examples of readers include, without limitation, BRD4, YEATS2, and PWWP. Examples of histone/DNA modifying enzymes include, without limitation, NSD2, JMJD2A, CARM1, MLL1, DOT1L, EZH2, and DNMT3A/B. Examples of chromatin regulatory complexes include, without limitation, RNA Polymerase II, SMARCA2, and ACF.

The detection reagent used in the methods of the invention may be any agent that specifically recognizes and binds to a histone, histone modification, DNA, DNA modification, or chromatin associated protein of interest present in a target epitope. In some embodiments, the detection reagent is an antibody or antibody fragment directed towards the epitope. The antibody or fragment thereof may be a full-length immunoglobulin molecule, an Fab, an Fab′, an F(ab)′₂, an scFv, an Fv fragment, a nanobody, a VHH or a minimal recognition unit. The detection reagent may be an aptamer or a non-immunoglobulin scaffold such as an affibody, an affilin molecule, an AdNectin, a lipocalin mutein, a DARPin, a Knottin, a Kunitz-type domain, an Avimer, a Tetranectin or a trans-body. The detection reagent may be a chromatin binding protein, e.g., a chromatin reader.

In some embodiments, the detection reagent may further comprise a binding moiety that may be used to isolate the released chromatin fragments. Examples of binding moieties and their binding partners include, without limitation, biotin with avidin or streptavidin, a nano-tag with streptavidin, glutathione with glutathione transferase, an antigen/epitope with an antibody, polyhistidine with nickel, a polynucleotide with a complementary polynucleotide, an aptamer with its specific target molecule, or Si-tag and silica. In some embodiments, the binding moiety is linked to the detection reagent and/or detection reagent binding protein. Alternatively, the digested chromatin fragments may be detected using a second detection reagent that binds to a second target epitope on the chromatin fragment. This antibody could be bound to a solid support such as a bead or a well of a microtiter plate and detected using any method known in the art (e.g., ELISA). For example, a first detection regent could be targeted to chromatin via an antibody to H3K4me3, followed by targeted cleavage by pAG-MNase. The cleaved chromatin fragments could then be captured on the surface of a plate coated with a second detection reagent that is specific for a second epitope associated with the cleaved chromatin fragments (e.g., methylated DNA or nucleosome) and detected by ELISA.

The methods of the invention may comprise measuring the total amount of more than one chromatin target epitope and a separate detection reagent that binds each chromatin target epitope is used. In some embodiments, 2, 3, 4, 5, 6, 7, 8, 9, or 10 or more different chromatin target epitopes are measured.

The detection reagent-binding reagent may be any reagent that specifically binds the detection reagent. In some embodiments, the detection reagent-binding agent is Protein A or a fragment thereof and/or Protein G or a fragment thereof (e.g., pAG). In some embodiments, the detection reagent-binding agent is a fusion protein comprising the inactive enzyme. In some embodiments, the detection reagent-binding reagent is micrococcal nuclease fused to Protein A or a fragment thereof and/or Protein G or a fragment thereof (e.g., pAG-MNase).

In some embodiments, the inactive enzyme is a nuclease, e.g., micrococcal nuclease. The inactive enzyme may be activated by addition of any suitable reagent, e.g., Ca²⁺ for micrococcal nuclease.

In some embodiments, the sample may be mechanically or enzymatically fragmented before and/or after enzyme activation.

In some embodiments, the biological sample is treated with a nuclease to standardize the DNA length of chromatin that is captured by a detection reagent. The uniform length of the DNA may be about 20-170 nucleotides, e.g., about 20-60 nucleotides, e.g., about 20-100 nucleotides, e.g., about 140-160 nucleotides, e.g., about 150 nucleotides. In some embodiments, the nuclease may be micrococcal nuclease. In some embodiments, nuclease treatment can be performed before and/or after nucleosome capture. Targeted digestion is key to the success of this assay, as readout is based on DNA quantification. If samples are not uniformly digested, then they will likely have low reproducibility and/or sensitivity.

Quantifying the amount of DNA associated with the released chromatin fragments may be carried out by digesting proteins (e.g., using proteinase K, RNase) followed by measuring the amount of DNA in the sample. In some embodiments, DNA is quantified using an intercalating dye, wherein the amount of fluorescence (i.e., DNA concentration) is proportional to the amount of fragmented DNA present. In some embodiments, the intercalating dye is PicoGreen® ((2-(n-bis-(3-dimethylaminopropyl)-amino)-4-(2,3-dihydro-3-methyl-(benzo-1,3-thiazol-2-yl)-methylidene)-1-phenyl-quinolinium)).

In some embodiments, recombinant modified nucleosomes carrying a target epitope (e.g., histone PTM, DNA modification, or chromatin associated protein) are used as standards for assay quantification, wherein nucleosomes are added to separate wells at a single concentration or a range of concentrations, treated similar to samples, and used as a reference standard to quantify nucleosome levels in samples. Suitable recombinant modified nucleosomes may be those described in, e.g., WO 2019/169263, incorporated by reference herein in its entirety.

A further aspect of the invention relates to kits for carrying out the methods of the invention. The kits may be specific for cell-based methods or cell-free methods. The kits may comprise, without limitation, targeting enzyme (e.g., pAG-MNase), detection reagent (e.g., chromatin targeting antibody), buffers, cell permeabilizer, beads for solid support, intercalating dye, positive and negative control antibodies, nucleosomes for reference standards, buffers, containers, instructions for carrying out the methods, etc.

Having described the present invention, the same will be explained in greater detail in the following examples, which are included herein for illustration purposes only, and which are not intended to be limiting to the invention.

### Examples

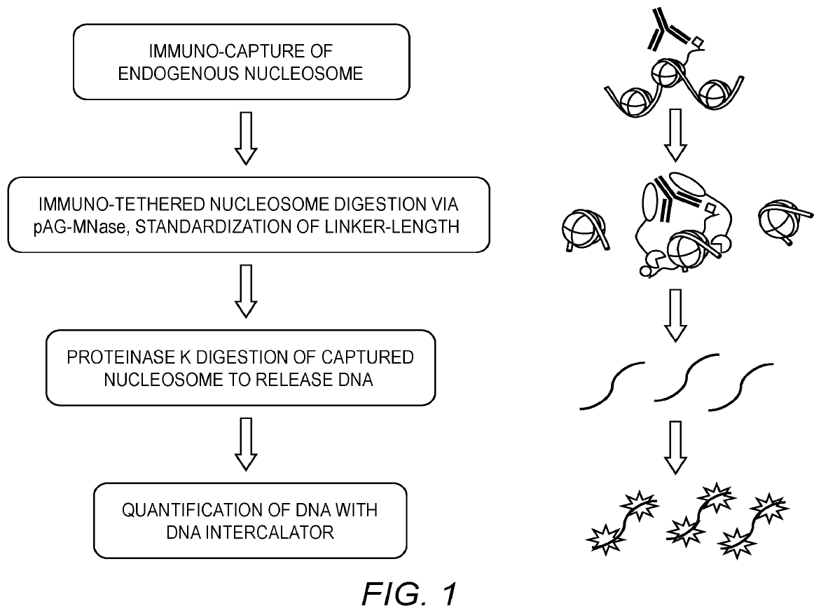

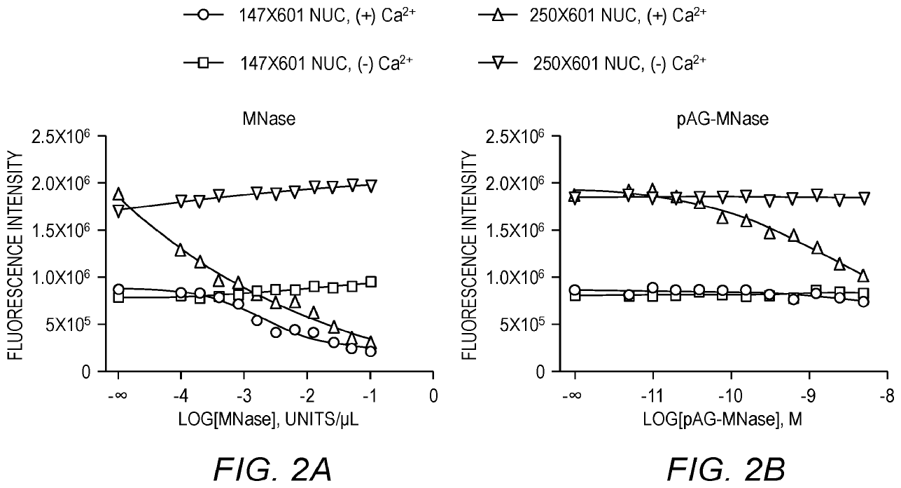

Example 1: Immuno-targeted cleavage by pAG-MNase is a critical innovation to the present invention, providing a highly efficient method to digest target chromatin into mononucleosomes (i.e., ˜150 bp) for reliable quantification of various chromatin elements, including histone PTMs and proteins that are directly or indirectly associated with chromatin (FIGS. 2A-2B). These assays can be used to analyze nucleosomes in situ (i.e., in intact cells) or in biological fluids (e.g., plasma, urine, stool, synovial fluid, etc.). Of note, pAG-MNase can also digest chromatin targets that directly bind DNA into subnucleosomal fragments (e.g., <150 bp), as DNA that is in direct contact with the chromatin interactor is shielded from digestion by pAG-MNase (or other nucleases). State of the art assays typically use MNase to digest samples into mononucleosome fragments. However, this approach is very challenging to optimize, as under-digestion fails to provide consistent mononucleosome preparations and over-digestion can lead to nucleosome instability, resulting in the disassociation of DNA from the histone octamer and subsequent digestion of the “free” DNA by MNase. By contrast, targeted cleavage using pAG-MNase allows for the MNase concentration at a given genomic loci to be more finely tuned. This results in consistent cleavage of DNA surrounding the target into mononucleosomes (or subnucleosome fragments in the case of chromatin associated protein targets) and limited over-digestion. Here, we compared the digestion of recombinant nucleosomes carrying no linker DNA (e.g., 147 bp×601 Nuc) or linker DNA (e.g., 250×601 Nuc) using MNase or pAG-MNase followed by DNA detection using our innovative sample processing workflow (see FIG. 1). For these experiments, the nucleosomes contained di-methylation at histone H3 lysine 4 (H3K4me2) to enable antibody targeting using pAG-MNase. As controls, we also assayed unassembled DNA (147 bp×601 DNA and 250 bp×601) to compare digestion between nucleosomes and free DNA as well as performed+/−calcium (MNase co-factor) to ensure that changes in DNA signal were dependent on MNase activity. Protein A beads bound to an anti-H3K4me2 antibody were used to capture H3K4me2 nucleosomes assembled with either 147 bp or 250 bp DNA. We then titrated the concentration of MNase or pAG-MNase against the captured nucleosome samples (147 bp or 250 bp) or DNA sample controls (147 bp or 250 bp). Following 30 minutes of enzyme activation, nucleosomes were treated with proteinase K to remove histone proteins, followed by PicoGreen©. DNA concentration was quantified by PicoGreen® fluorescence. As expected, we observed shorter fragments of DNA as the concentration of MNase increased (FIG. 2A). Notably, the DNA length of both the 250 bp and 147 bp nucleosomes became shorter than 147 bp, suggesting that the nucleosome was being over digested (and likely falling apart in solution). By contrast, antibody-targeted cleavage by pAG-MNase did not result in fragments shorter than 147 bp, suggesting that nucleosome integrity was maintained (FIG. 2B). Further we found that digestion of the 250×601 nucleosome DNA was slower and reached ˜150 bp after 30 minutes of digestion. These results show that targeted immuno-cleavage results in different digestion kinetics vs. untethered MNase. Our assay leverages this advantage to reliably generate mononucleosomes that contain a standardized nucleosomal DNA length of ˜150 bp. While these assays were performed using recombinant nucleosomes, these assays are also useful for broad range of samples, including intact cells or biological fluids. Further, these assays could use any antibody that targets a chromatin feature, such as antibodies for transcription factors, chromatin enzymes, or other chromatin interactor proteins.

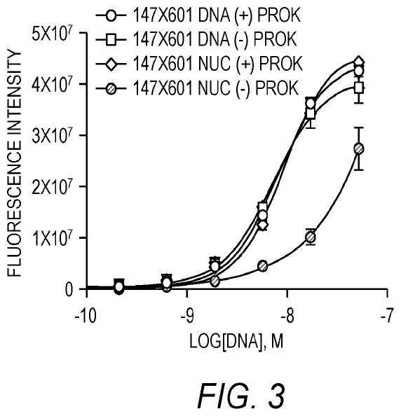

Example 2: Removal of histone proteins from nucleosome DNA improves assay signal (FIG. 3). Targeted cleavage of the sample using pAG-MNase generates mononucleosomes comprised of ˜150 bp DNA and a histone octamer. Here, we asked the impact of the histone octamer (or other DNA-binding proteins) on fluorescence by DNA intercalators. For these studies, we treated mononucleosome (147 bp×601 Nuc)+/−proteinase K, which digests all proteins, including histones. Following histone digestion, samples were treated with PicoGreen® and quantified using PicoGreen® fluorescence. As controls, we also treated 147 bp×601 DNA. We found that the fluorescence signal was dramatically reduced on nucleosomes without proteinase K treatment. This is likely due to the inability of PicoGreen® to fully associate with DNA when in a nucleosome context. Thus, treatment with proteinase K is a key step to improve assay signal; however, it is not essential to successfully perform the assay, as assay signal can still be detected in undigested samples.

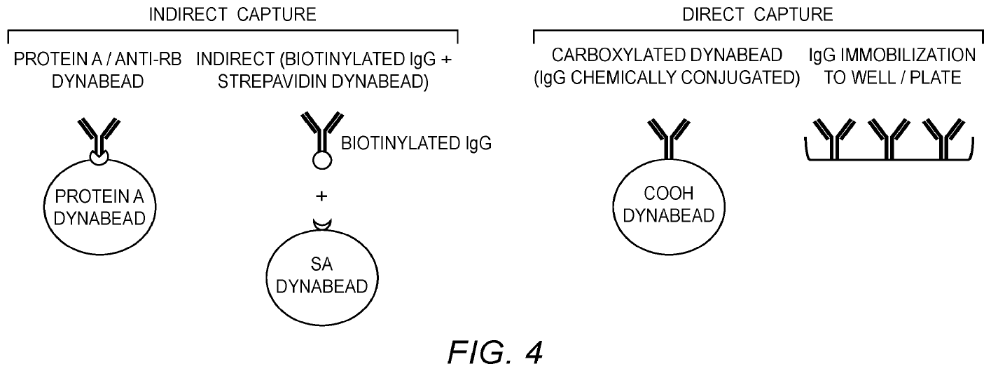

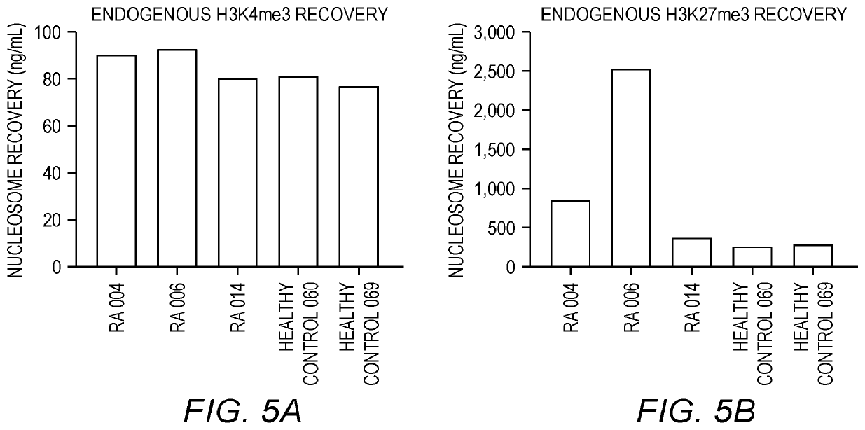

Example 3: Validation of assay to measure endogenous cell-free nucleosomes from human plasma. There are various assay configurations that can be used to capture nucleosomes from biological fluids (FIG. 4). Some methods indirectly couple antibodies to beads. For example, antibodies can be bound to protein A-coated beads. Alternatively, biotinylated antibodies can be bound to streptavidin-coated beads. Some methods directly couple antibodies to a solid support. For example, antibodies can be chemically linked to a bead. Alternatively, antibodies can be immobilized directly to a well or wells of a microtiter plate. Here we tested a “direct” assay configuration (antibodies bound to a microtiter plate) to test the utility of our assay to measure changes in histone PTMs between healthy and disease plasma samples. For this study, we measured two different histone modifications, H3K4me3 and H3K27me3, and measured the global abundance of these marks in patients with rheumatoid arthritis and healthy controls. H3K4me3 is a marker of active promoters and is found on ˜0.5% of nucleosomes genome-wide. H3K27me3 is a marker of heterochromatin (i.e., silenced genes) and is found on ˜20% of nucleosomes genome-wide. The distinct functions and global distribution of these marks make them an excellent test set for assay validation. The assay was performed as follows: i) antibodies were bound to a 96-well plate, similar to a standard ELISA; ii) diluted plasma samples were added to each well; iii) recombinant nucleosomes carrying H3K4me3 or H3K27me3 were added to different wells to generate a standard curve for assay quantification; iv) pAG-MNase was added to each well and activated to standardize captured nucleosome length; v) samples were digested with proteinase K and treated with PicoGreen®, and DNA was quantified using PicoGreen® fluorescence. We observed no difference in H3K4me3 levels when comparing RA patient plasma and controls (FIG. 5A). However, we observed an increase in the amount of H3K27me3 in some of the RA samples vs. the controls (FIG. 5B). These data show that our assay has the sensitivity to detect changes in histone PTM levels on nucleosomes directly from plasma samples.

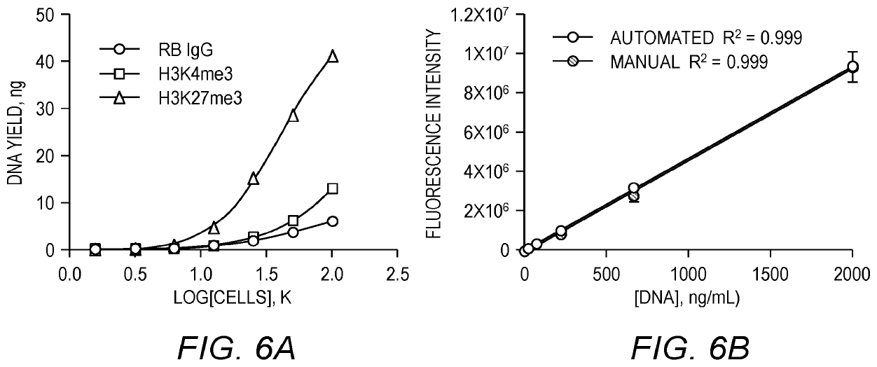

Example 4: Validation of assay to measure global levels of histone PTMs directly from cells. Measuring histone PTMs on nucleosomes from cells is challenging due to the need to isolate mononucleosomes from the intact chromatin. Current methods use sonication or enzymatic digestion, which is difficult to optimize and is time consuming. Here we demonstrated the utility of our assay to measure histone PTMs directly from intact cells. These assays could be useful for measuring changes in histone PTMs (or other chromatin regulators) in response to cellular treatment, such as stimulation or drug treatment. Indeed, these assays are easy to automate, providing a high-throughput solution for diagnostic, personalized medicine, or drug development applications. For these studies, various numbers of K562 cells were bound to lectin coated beads. Next, cells were permeabilized with digitonin and incubated overnight with antibodies to H3K4me3 or H3K27me3 (anti-IgG was used as a control). Following washing, pAG-MNase was added to samples and activated to cleave nearby nucleosomes, releasing them into solution. Beads (conjugated to cells) were pelleted, and cleaved DNA fragments were purified using carboxylated beads. Finally, DNA fragments were treated with proteinase K, PicoGreen®, and then quantified using PicoGreen® fluorescence. As expected, we observed an increase in signal for H3K4me3 and H3K27me3 as the number of cells increased (FIG. 6A). Importantly, we observed high signal for H3K27me3 vs. H3K4me3 as this mark is more abundant in the cell; moreover, both H3K4me3 and H3K27me3 generated greater signal than our IgG control. These assays were performed using a liquid handler (Tecan Freedom EVO), demonstrating their compatibility with automation. As expected, we observed similar DNA quantification when using automated vs. manual workflows. FIG. 6B shows a head-to-head comparison of DNA concentration levels using our H3K4me3 nucleosome standard curve. Of note, current state of the art methods are not readily compatible with automation, highlighting a major advantage of the present invention.

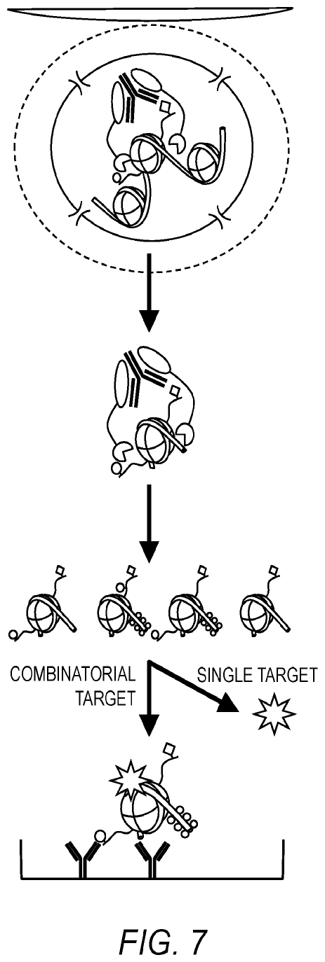

Example 5: The cell-based assay from example 4 can be modified to also detect combinatorial modifications by adding an additional capture step following cleavage by pAG-MNase (FIG. 7). For example, cleaved nucleosomes (or subnucleosomal fragments in the case when targeting chromatin associated proteins) could be captured by a second antibody to a second target also found on the chromatin fragment. This antibody could be bound to a solid support, such as a plate or bead, to allow for singly modified fragments to be washed away prior to proteinase K digestion and detection of dually modified chromatin fragments by PicoGreen®. For example, a first antibody is directed to H3K9me3. Following targeted cleavage, a second antibody to H3K36me3 is used to capture the cleaved fragments. This second antibody is bound to a bead or microtiter plate, allowing for nucleosomes with only H3K36me3 to be washed away. Finally, the H3K9me3/H3K36me3 modified nucleosomes are treated with proteinase K and quantified via PicoGreen© fluorescence. Alternatively, the second target could be detected using a standard sandwich ELISA approach.

### REFERENCES

Baranello, L., F. Kouzine, S. Sanford and D. Levens (2016). “ChIP bias as a function of cross-linking time.” Chromosome Res 24(2): 175-181.
Chereji, R. V., T. D. Bryson and S. Henikoff (2019). “Quantitative MNase-seq accurately maps nucleosome occupancy levels.” Genome Biol 20(1): 198.
Grzybowski, A. T., Z. Chen and A. J. Ruthenburg (2015). “Calibrating ChIP-Seq with Nucleosomal Internal Standards to Measure Histone Modification Density Genome Wide.” Mol Cell 58(5): 886-899.
Grzybowski, A. T., R. N. Shah, W. F. Richter and A. J. Ruthenburg (2019). “Native internally calibrated chromatin immunoprecipitation for quantitative studies of histone post-translational modifications.” Nat Protoc 14(12): 3275-3302.
Henikoff, S. and J. G. Henikoff (2020). “Profiling the epigenome at home.” bioRxiv: 2020.2004.2015.043083.
Meers, M. P., T. D. Bryson, J. G. Henikoff and S. Henikoff (2019). “Improved CUT&RUN chromatin profiling tools.” Elife 8.
Schmid, M., T. Durussel and U. K. Laemmli (2004). “ChIC and ChEC; genomic mapping of chromatin proteins.” Mol Cell 16(1): 147-157.
Shah, R. N., A. T. Grzybowski, E. M. Cornett, A. L. Johnstone, B. M. Dickson, B. A. Boone, M. A. Cheek, M. W. Cowles, D. Maryanski, M. J. Meiners, R. L. Tiedemann, R. M. Vaughan, N. Arora, Z. W. Sun, S. B. Rothbart, M. C. Keogh and A. J. Ruthenburg (2018). “Examining the Roles of H3K4 Methylation States with Systematically Characterized Antibodies.” Mol Cell 72(1): 162-177 e167.
Skene, P. J., J. G. Henikoff and S. Henikoff (2018). “Targeted in situ genome-wide profiling with high efficiency for low cell numbers.” Nat Protoc 13(5): 1006-1019.
Thalin, C., K. Aguilera, N. W. Hall, M. R. Marunde, J. M. Burg, A. Rosell, M. Daleskog, M. Mansson, Y. Hisada, M. J. Meiners, Z. W. Sun, M. F. Whelihan, M. A. Cheek, S. A. Howard, S. Saxena-Beem, D. F. Noubouossie, N. S. Key, S. Z. Sheikh, M. C. Keogh, M. W. Cowles, S. Lundstrom, N. Mackman, H. Wallen and A. L. Johnstone (2020). “Quantification of citrullinated histones: Development of an improved assay to reliably quantify nucleosomal H3Cit in human plasma.” J Thromb Haemost.
van Steensel, B. and S. Henikoff (2000). “Identification of in vivo DNA targets of chromatin proteins using tethered dam methyltransferase.” Nat Biotechnol 18(4): 424-428.
Xi, Y., J. Yao, R. Chen, W. Li and X. He (2011). “Nucleosome fragility reveals novel functional states of chromatin and poises genes for activation.” Genome Res 21(5): 718-724.
Zhang, Z. and B. F. Pugh (2011). “High-resolution genome-wide mapping of the primary structure of chromatin.” Cell 144(2): 175-186.

The foregoing is illustrative of the present invention, and is not to be construed as limiting thereof. The invention is defined by the following claims, with equivalents of the claims to be included therein.

1. A method for measuring the total amount of a chromatin target epitope in a biological sample, the method comprising:
a. obtaining a biological sample comprising chromatin;
b. contacting the chromatin with a detection reagent that binds a chromatin target epitope to form a complex;
c. contacting the complex with a detection reagent-binding agent linked to an inactive enzyme;
d. activating the enzyme to digest the chromatin in a targeted manner to release chromatin fragments;
e. trimming DNA associated with the released chromatin fragments to produce a uniform length;
f. isolating the released chromatin fragments; and
g. quantifying the amount of DNA associated with the released chromatin fragments;
thereby measuring the total amount of the chromatin target epitope in the biological sample;
wherein the detection reagent is the only chromatin binding agent used in the method.

2. A method for measuring the total amount of a chromatin target epitope in a biological sample, the method comprising:
a. obtaining a biological sample comprising chromatin;
b. obtaining reference nucleosomes comprising the chromatin target epitope to form a reference sample;
c. contacting each of the chromatin and the reference sample with a detection reagent that binds a chromatin target epitope to form a complex;
d. contacting the complex with a detection reagent-binding agent linked to an inactive enzyme;
e. activating the enzyme to digest the chromatin and the reference sample in a targeted manner to release chromatin fragments;
f. trimming DNA associated with the released chromatin fragments to produce a uniform length;
g. isolating the released chromatin fragments from the chromatin and the reference sample; and
h. quantifying the amount of DNA associated with the released chromatin fragments from the chromatin and the reference sample;
thereby quantifying the level of the chromatin target epitope in the biological sample;
wherein the detection reagent is the only chromatin binding agent used in the method.

3. A method for measuring the total amount of a chromatin target epitope in a biological sample, the method comprising:
a. obtaining a biological sample comprising chromatin;
b. binding the biological sample to a solid support;
c. contacting the chromatin with a detection reagent that binds a chromatin target epitope to form a complex;
d. contacting the complex with a detection reagent-binding agent linked to an inactive enzyme;
e. activating the enzyme to digest the chromatin in a targeted manner to release chromatin fragments from the solid support;
f. trimming DNA associated with the released chromatin fragments to produce a uniform length;
g. isolating the released nucleosomes; and
h. quantifying the amount of DNA associated with the released chromatin fragments;
thereby measuring the total amount of the chromatin target epitope in the biological sample;
wherein the detection reagent is the only chromatin binding agent used in the method.

4. A method for measuring the total amount of a chromatin target epitope in a biological sample, the method comprising:
a. obtaining a biological sample comprising chromatin;
b. obtaining reference nucleosomes comprising the chromatin target epitope to form a reference sample;
c. binding the biological sample and the reference sample to a solid support;
d. contacting each of the chromatin and the reference sample with a detection reagent that binds a chromatin target epitope to form a complex;
e. contacting the complex with a detection reagent-binding agent linked to an inactive enzyme;
f. activating the enzyme to digest the chromatin and the reference sample in a targeted manner to release chromatin fragments from the solid support;
g. trimming DNA associated with the released nucleosomes to produce a uniform length;
h. isolating the released chromatin fragments from the chromatin and the reference sample; and
i. quantifying the amount of DNA associated with the released chromatin fragments from the chromatin and the reference sample;
thereby quantifying the level of a nucleosome target epitope in the biological sample;
wherein the detection reagent is the only chromatin binding agent used in the method.

5-8. (canceled)

9. The method of claim 1, wherein the biological sample is a cell, a nucleus isolated from a cell, or chromatin isolated from a cell.

10-11. (canceled)

12. The method of claim 1, wherein the biological sample is a biopsy or a biological fluid.

13. (canceled)

14. The method of claim 1, wherein the chromatin target epitope is a histone modification, histone variant, histone mutation, unmodified histone, unmodified DNA, DNA modification, and/or a protein that indirectly or directly binds chromatin.

15. The method of claim 1, where the biological sample is from a subject having a disease or disorder associated with changes in one or more histone post-translational modifications and/or DNA modifications.

16. The method of claim 14, wherein the histone and/or DNA modification is selected from the group consisting of N-acetylation of serine and alanine; phosphorylation of serine, threonine and tyrosine; N-crotonylation, N-acylation of lysine; N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine, ADP-ribosylation of arginine, aspartic acid and glutamic acid; oncogenic K-to-M mutations, 5-methylcytosine, 5-hydroxymethylcytosine, 5-formylcytosine, 5-carboxylcytosine, 3-methylcytosine, 5,6-dihydrouracil, 7-methylguanosine, xanthosine, inosine, and any combination thereof.

17. The method of claim 1, wherein the method comprises measuring the total amount of more than one nucleosome target epitope and a separate detection reagent that binds each nucleosome target epitope is used.

18. The method of claim 1, wherein the detection reagent is an antibody or fragment thereof, aptamer, nanobody, or chromatin associated protein.

19. The method of claim 1, wherein the detection reagent comprises a binding moiety.

20. (canceled)

21. The method of claim 19, wherein the binding moiety is used to isolate the released nucleosomes.

22. The method of claim 1, wherein the detection reagent is linked to a solid support.

23. The method of claim 1, wherein the uniform length of nucleosome-associated DNA is about 20 to about 170 nucleotides.

24. The method of claim 1, wherein the enzyme is a nuclease, such as micrococcal nuclease.

25. (canceled)

26. The method of claim 1, wherein the detection reagent-binding agent is a fusion protein comprising the inactive enzyme.

27. The method of claim 1, wherein the detection reagent-binding agent is pAG.

28. The method of claim 1, wherein the amount of DNA is quantitated using an intercalator dye, such as (2-(n-bis-(3-dimethylaminopropyl)-amino)-4-(2,3-dihydro-3-methyl-(benzo-1,3-thiazol-2-yl)-methylidene)-1-phenyl-quinolinium).

29. (canceled)

30. The method of claim 1, wherein the sample is mechanically or enzymatically fragmented before enzyme activation and/or after enzyme activation.

31. (canceled)

32. The method of claim 1, wherein the sample is treated with one or more additional enzymes after the digestion with enzyme, such as RNase and/or Proteinase K.

33. (canceled)

34. The method of claim 2, wherein the reference nucleosomes are present in the reference sample at a single concentration.

35. The method of claim 2, wherein the reference nucleosomes are present in multiple reference samples at different concentrations.

36. The method of claim 3, wherein the solid support is a magnetic bead.
